(19) United States
(12) Patent Application Publication
(10) Pub. No.: US 2007/0185369 A1
(43) Pub. Date: Aug. 9, 2007

(54) Cardiac assist device and method

(76) Inventors: Mahmood Mirhoseini, Germantown, WI (US); Mary M. Cayton, Germantown, WI (US); Valerie Checkanov, Franklin, WI (US); Nosrart-o-llah Mokhtari, Tehran (IR); Ebrahim Afjei, Tehran (IR); Hossien Fajani, Tehran (IR); Mohammad Barandack, Tehran (IR)

Correspondence Address: MICHAEL BEST & FRIEDRICH, LLP, 100 E WISCONSIN AVENUE, Suite 3300, MILWAUKEE, WI, 53202, US

(21) Appl. No.: 11/347,821

(22) Filed: Feb. 3, 2006

(51) Int. Cl. A61M 1/10; A61N 1/362

(52) U.S. Cl. 600/16; 607/9

(57) ABSTRACT

Cardiac assist device fully implantable within a patient and method of assisting the beating of the patient's heart. The cardiac assist device can include a cardiac jacket that wraps around at least a portion of the heart and a fluid reservoir coupled to the cardiac jacket. The cardiac assist device can include a pump that provides fluid to the cardiac jacket from the fluid reservoir and a motor coupled to the pump. A speed of the motor can control a fluid volume in the cardiac jacket. The cardiac assist device can include a pacemaker coupled to the motor. The pacemaker can control the speed of the motor based on cardiac parameters.

### BACKGROUND OF THE INVENTION

Heart failure as a result of end stage coronary artery disease or other cardiac conditions is an increasingly prevalent problem. The costs associated with frequent hospital admissions, medications, and outpatient visits are staggering. Heart failure currently accounts for one million hospitalizations annually in the United States. There are approximately four million people diagnosed with heart failure in the country, and with an increasingly aging population, the absolute number of patients is increasing progressively. Despite advances in both diagnostic methods and treatment alternatives, the mortality of late stage disease in symptomatic patients approaches 50 percent at one year. For those with mild disease, the mortality rate is 50 percent within 4-5 years.

The causes of heart failure are many, but the fundamental defect is the same. There is an imbalance between the output of blood from the heart and the demand of the body for that blood output. The imbalance of blood flow is associated with water and salt retention, resulting in central and/or peripheral edema. A failing heart undergoes structural changes, dialates, and assumes a spherical shape, rather than the normal elliptical shape. As a result of these spatial changes, the heart valves become incompetent. A spherical heart is a dysfunctional heart. The elliptical heart is mechanically more efficient and more stable electrically. The loss of elasticity in the failing ventricle means the heart is incapable of providing the necessary pumping function to accommodate body needs.

Although a number of invasive procedures have been employed to remedy the condition, and new medications have been developed, a fully satisfactory method of treating this condition has not been discovered. Approaches to the treatment of heart failure have included medical treatment only, intra-aortic balloon pump, heart transplantation, cardiomyoplasty, left ventricular excision, and wrapping the heart.

Medication is only effective on a temporary basis, and because of the strong effects of these medicines, there are often major side effects. Medication can only be used for relatively minor incidences of heart failure. In severe heart failure, medications have little or no effect. In advanced heart failure, there is not a medication existing which will force the myocardium with no contractile strength to perform. In less severe cases, the increase in contractile strength with medical therapy is only 15 percent.

Intra-aortic balloon counterpulsation (IABP) can only be used on a temporary basis. Inflation and deflation of the balloon, usually inserted percutaneously through the femoral artery, in the aorta increases diastolic blood flow to the coronary arteries. Improved myocardial blood flow increases the pumping function of the left ventricle. In general, an increase of 10-20 percent in contractile function can be achieved. Morbidity increases with each day the balloon is in place, and includes obstruction to blood flow to the affected limb, coagulopathy, infection, and malfunction of the inflation/deflation function of the balloon.

Heart transplant, as an option, is limited by the number of donor hearts available, and by the age and co-morbidity or disease present in the medical condition of the recipient. There is the consideration of life long immune suppression therapy, and frequent follow-up treatments. The costs of medications and treatments are very high. Transplant rejection is always a consideration. Arteriosclerotic disease of the coronary arteries in the transplanted heart is also known to affect long-term results.

Cardiomyoplasty requires an extensive surgical procedure. The latissimus dorsi muscle is dissected, lifted, and wrapped around the heart. Electrical stimulation of the implanted muscle results in contraction, creating pressure on the ventricle and thereby increasing cardiac output. The procedure is still experimental. Because of the complexity and extent of the surgical procedure, it is only suitable for very severe cases of pump (heart) failure. The pacemakers required for electrical stimulation of the muscle are costly. Patients require extensive follow-up and care following the procedure.

Excision of non-contractile left ventricular muscle (Batista Procedure), with the goal of increasing cardiac output is controversial. As with cardiomyoplasty, results are still open to debate.

A suitable artificial heart has yet to be developed, in spite of years of experimentation with various models. The biggest obstacles are the incompatibility of the blood with the artificial heart which causes coagulation disturbances, the external systems required to effect the pumping mechanism that limit patient activity, and the morbidity associated with implanting the systems. Temporary assist systems, designed for use until a suitable donor heart can be found for transplantation, have the same drawbacks as the artificial heart.

A number of mechanical techniques for increasing cardiac output, and assisting the failing heart, include compressing the outer epicardial surface of the heart. Various models of cardiac wraps have been proposed. In general, the cardiac wraps are inflated and deflated cyclically, in response to cardiac output parameters. In all instances of existing technology, cardiac output and function is monitored and regulated externally by mechanical means. The techniques include wrapping the heart with a mesh or biocompatible material, and applying pressure to the ventricle. Direct cardiac compression (DCC) techniques have focused mainly on the left ventricular (LV) systolic and diastolic pressure. The technique does not increase diastolic function. The end diastolic pressure-volume relationship (EDPVR) is altered, and right ventricular (RV) diastolic function is impaired. In addition, both LV and RV loading are required for this to be effective. Septal motion, ventricular wall motion, chamber dynamics, and overall cardiac function are not considered.

Dynamic mechanical assist devices for the heart include wrapping the heart with a two-layer membrane. The inner membrane conforms to the exterior surface of the heart throughout systole and diastole by means of a mechanical control system that inflates and deflates the inner wrap. This method provides enhanced support to the failing heart by closer regulation of cardiac function. A number of devices, from the complex to the simple, have been described using the liner system for allowing compression and relaxation of the cardiac muscle. The applications require tubes connected to the compression device to extend externally from the body to access ports. Management of cardiac parameters, by increasing or decreasing the amount of fluid in the liner, is done mechanically. To acquire full knowledge of cardiac parameters, direct pressure readings, echocardiographic management, and other expensive and time-consuming techniques are required. In the above methods, fluid is added or removed from the jacket or liner by mechanical means.

### SUMMARY OF THE INVENTION

Embodiments of the invention provide a cardiac assist device fully implantable within a patient to assist the beating of the patient's heart. The cardiac assist device can include a cardiac jacket that wraps around at least a portion of the heart and a fluid reservoir coupled to the cardiac jacket via an inflow canal and an outflow canal. The cardiac assist device can include a pump that provides fluid to the cardiac jacket from the fluid reservoir and a motor coupled to the pump. A speed of the motor can control a fluid volume in the cardiac jacket. The cardiac assist device can include a pacemaker coupled to the motor. The pacemaker can control the speed of the motor based on one or more cardiac parameters.

Embodiments of the invention provide a method of assisting the beating of a patient's heart. The method can include fully implanting a cardiac assist device within the patient, the cardiac assist device including a cardiac jacket, a fluid reservoir, a pump, a motor, and a pacemaker. The method can include wrapping the cardiac jacket around at least a portion of the heart and pumping fluid to the cardiac jacket from the fluid reservoir. The method can include controlling a speed of the motor based on at least one cardiac parameter in order to control a fluid volume in the cardiac jacket.

### BRIEF DESCRIPTION OF THE DRAWINGS

FIG. 1 is a schematic view of a cardiac assist device according to one embodiment of the invention.

FIG. 2 is a schematic view of a cardiac assist device according to another embodiment of the invention.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect.

In addition, embodiments of the invention include both hardware and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

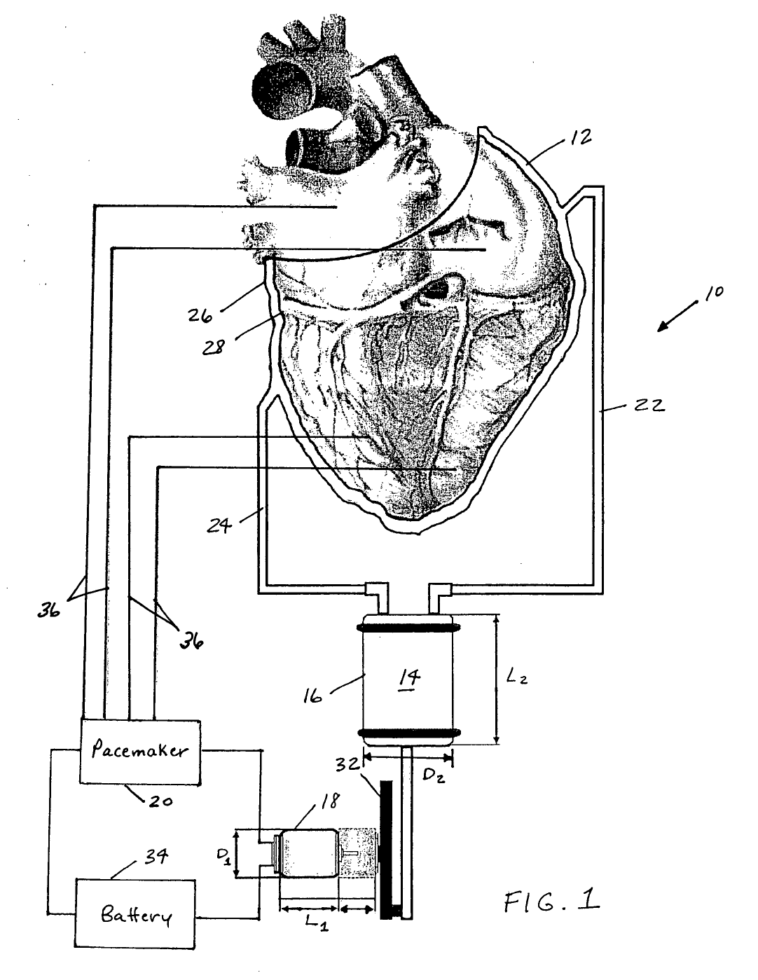

FIG. 1 illustrates a cardiac assist device 10 according to one embodiment of the invention. The cardiac assist device 10 can be fully implantable within a patient to assist the beating of the patient's heart. The cardiac assist device 10 can include a cardiac jacket 12, a fluid reservoir 14, a pump 16, a motor 18, and pacemaker 20. The cardiac jacket 12, the fluid reservoir 14, the pump 16, the motor 18, and the pacemaker 20 can be fully implantable subcutaneously in either the left or right chest or the upper abdomen.

The cardiac jacket 12 can wrap around at least a portion of the patient's heart. The fluid reservoir 14 can be coupled to the cardiac jacket 12 via an inflow canal 22 and an outflow canal 24. The pump 16 can provide fluid to the cardiac jacket 12 from the fluid reservoir 14. The motor 18 can be coupled to the pump 16. The speed of the motor 18 can control a fluid volume in the cardiac jacket 12. The pacemaker 20 can be coupled to the motor 18. The pacemaker 20 can control the speed of the motor 18 based on one or more cardiac parameters. In some embodiments, the cardiac jacket 12, the fluid reservoir 14, the pump 16, the motor 18, and the pacemaker 20 are each implantable in the patient and do not require any external ports, which can be sources of infection. In some embodiments, none of the cardiac jacket 12, the fluid reservoir 14, the pump 16, the motor 18, and the pacemaker 20 interface with a patient's blood, which could cause coagulation problems.

The inflow canal 22 and the outflow canal 24 can be connected to the fluid reservoir 14, which can be connected to the pump 16 and controlled by the motor 18. In some embodiments, the inflow canal 22 and the outflow canal 24 can each include a check valve. A fluid space in the cardiac jacket 12 can be primed with fluid at about zero or a minimal pressure. The cardiac jacket 12 and the fluid reservoir 14 can each be constructed of at least one layer of material that is leak-proof, impermeable, and self-sealing. A prime volume of the fluid reservoir 14 can be predetermined based on a size of the patient and a degree of left ventricular dysfunction. The fluid reservoir 14 can include an additional fluid volume to adjust hemodynamics. The additional fluid volume can be about 10 percent to about 20 percent of the prime volume. A compression fluid volume can be added to the prime volume. The compression fluid volume can depend on a desired systolic pressure.

Some embodiments of the invention include a two-layer cardiac jacket 12, which can be inflated and deflated so that it assists both the left and right ventricle. The cardiac jacket 12 can include a non-distensible layer 26 and a compressible layer 28. The non-distensible layer 26 can be an external layer with respect to the heart and the compressible layer 28 can be an internal layer with respect to the heart. The cardiac jacket 12 can be constructed of biocompatible material including two layers. The internal compressible layer 28 can be flexible and able to be quickly inflated and deflated. The internal compressible layer 28 can be constructed of a material that does not fracture or lose strength with repeated inflation and deflation. Both the layers 26 and 28 can be leak proof, impermeable, and self-sealing in the event of puncture.

In some embodiments, the internal compressible layer 28 can have shape memory and does not stretch or lose its primary dimensions or maximum distensibility. Memory wires can be threaded through the internal compressible layer 28 of the jacket 12 both horizontally and vertically. This allows for consistency in the application of and the release of pressure in the internal membrane. Insertion of memory wires assists in compression, and reduces the energy required to achieve the level of compression desired once an optimal compression setting is achieved. Carbon nanotubes or ribbons, which are electrically conductive, can be embedded into the internal compressible layer 28. The nanotubes or ribbons can be used to shorten the response time for inflation and deflation, and also may be used to integrate information on cardiac parameters via telemetry.

In some embodiments, the cardiac jacket 12 can include two or more compartments. In one embodiment, the compartments can include a left ventricular compartment and a right ventricular compartment. The left ventricular compartment and the right ventricular compartment can be controlled independently with a delay (e.g., about 30 milliseconds to about 34 milliseconds between the left ventricle and the right ventricle). In one embodiment, the compartments can include an atrial compartment and a ventricular compartment. The jacket can also be compartmentalized to allow one to synchronize the function of right ventricle vs. left ventricle and atria vs. ventricles.

Compression pressure of the jacket 12 can respond to the circulatory needs of the body. A set volume of fluid from the reservoir 14 can be added to the prime volume in the jacket 12, depending on the systolic pressure of the body desired to be achieved. When fluid is added to the space between the layers 26 and 28 of the jacket 12, the internal compressible layer 28 can compress the ventricle, while the external non-compressible layer 26 can retain its shape and remain constant.

Some embodiments of the invention include a fully-implantable system for monitoring cardiac parameters and inflating or deflating the liner as necessary. The fully implantable, interactive, pulsatile, system can include the jacket 12, the fluid inflow canal 22, the outflow canal 24, the fluid reservoir 14, the pump 16, the motor 18, and the pacemaker 20, all of which can be programmed and/or recharged externally. Because of the small size, the entire system 10 can be implanted subcutaneously in either the left or right chest or the upper abdomen. All materials used can be biocompatible. Patients can be physically active with the device implanted.

The pump 16 can include a length of about three centimeters to about four centimeters and a diameter of about five centimeters. For example, using nanotechnology concepts, the pump 16 specifications can include a length L₂ of 3.4 cm and a diameter D₂ of 5 cm. The pump 16 can use exotic materials for the welded parts. For example, the pump 16 can be constructed of material including high purity thermoplastic. The pump 16 can include one or more impellars and a shaft constructed of a material including ceramic.

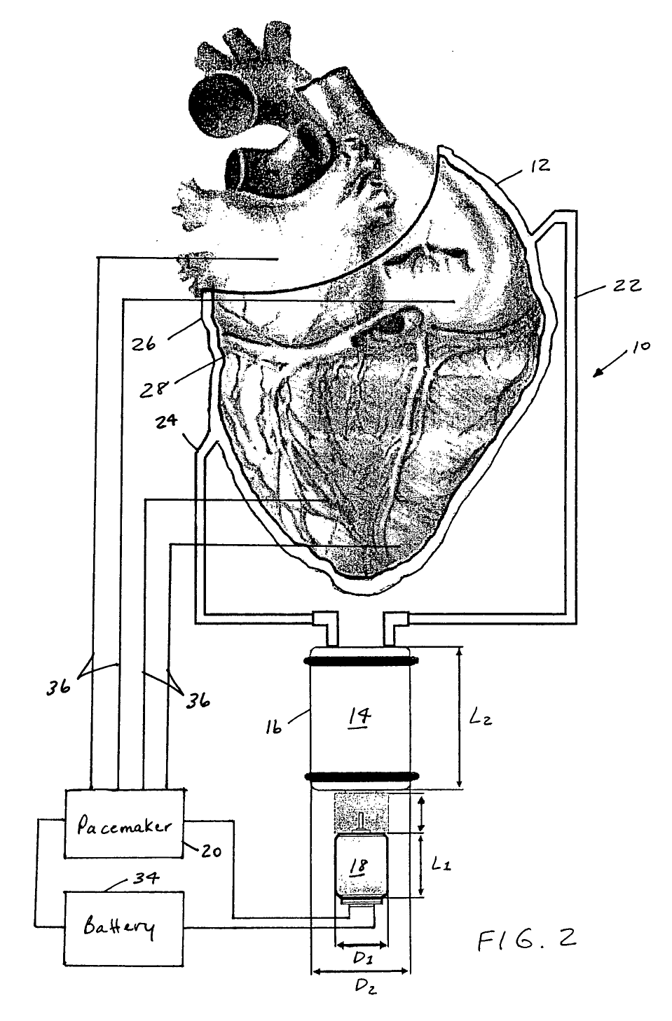

As shown in FIG. 1, a connector 32 can be coupled between the pump 16 and the motor 18 to change rotation of the motor 18 to linear movement of the pump 16. The connector 32 can include a wheel having a diameter approximately equal to a displacement of a shaft of the pump 16. However, as shown in FIG. 2, the pump 16 and the motor 18 can be arranged so that a connector 32 is not necessary.

In some embodiments, the motor 18 is a servo brushless direct current motor with a high starting torque and with a configuration to allow more space for coil winding. For example, the motor 18 can be a Series 1717 SR direct current micromotor with a precious metal commutator for use with a Series 16A spur gearhead, both manufactured by Faulhaber. The motor 18 can be powered by a rechargeable battery 34. In some embodiments, the battery 34 can be externally recharged by radio frequency through a coil external to the patient. In one embodiment, the battery charge can hold for about 1-2 hours of continuous operation. In one embodiment, the motor 18 can include a length L₁ of about 17 millimeters and a diameter D₁ of about 10 millimeters. The motor 18 can operate according to one or more of the following parameters: a normal voltage of about three Volts to about six Volts, a power output of about two Watts, an efficiency of about 69 percent, a rotor inertia of about 0.6 grams centimeters squared, and a maximum recommended speed of about 1000 revolutions per minute. The fluid volume to inflate and deflate the cardiac jacket 12 can be controlled by the speed of the motor 18. Changing the speed of the motor 18 and the amount of fluid delivered, can allow adjustment of systolic pressure and can augment the function of the ventricles. Response to changing hemodynamic parameters can be in real time.

In one embodiment, a pressure in the cardiac jacket 12 is about 100 millimeters of mercury when a compression volume is about 70 cubic centimeters. In one embodiment, the motor 18 and the pump 16 can deliver to or remove from the cardiac jacket 12 about 100 cubic centimeters of fluid per second.

The pacemaker 20 can regulate compression of the heart by the cardiac jacket 12. For example, a synchronized pacemaker can regulate pulsatility of the compressions. The pacemaker 20 can cause compression during a systolic phase in order to increase systolic blood pressure. The ventricle can be compressed during systole, thereby assisting the heart to increase the systolic blood pressure. The pacemaker 20 can regulate decompression of the heart by the cardiac jacket 12. The pacemaker 20 can cause decompression during a diastolic phase in order to at least partially assist the heart in the diastolic phase. During the diastolic phase of the cardiac cycle, fluid can return to the fluid reservoir 14 via the outflow canal 24, and thus, can also partially assist the heart in the diastolic phase of the cardiac cycle.

Synchronization by the pacemaker 20 can be based on dual-mode, dual-pacing, dual-sensing (DDD) pacing, biventricular pacing, and/or three-chamber synchronization pacing. Depending on the amount of cardiac support needed, the pacemaker 20 can regulate with a pulsation ratio of inflation and deflation of the jacket 12 of one to one, one to two, one to three, one to four, etc. Pulsation ratios of inflation and deflation of the jacket 12 can be adjusted on the basis of cardiac parameters, and the severity of the heart failure. A lower pulsation ratio can extend use of the rechargeable battery powering the motor 18 to between about two hours and about six hours.

The pacemaker 20 can monitor the heart with one or more leads 36 coupled to one or more of the right ventricle, the left ventricle, the right atrium, and the left atrium. The pacemaker 20 can include a processor that determines left ventricular cardiac parameters and right ventricular cardiac parameters. The cardiac parameters can include one or more of the following: left ventricular end diastolic pressure (LVEDP), left ventricular end systolic pressure (LVESP), right ventricular end diastolic pressure (RVEDP), right ventricular end systolic pressure (RVESP), left ventricular volume, right ventricular volume, cardiac tension, cardiac output, systolic blood pressure, diastolic blood pressure, and heart rate. The pacemaker 20 can respond to changes in the cardiac parameters by changing the inflation rate, the deflation rate, and/or the fluid volume. In some embodiments, the pacemaker 20 can continuously monitor and regulate cardiac hemodynamics in real time. The monitoring and regulating can be continuous and can immediately respond to changing cardiac hemodynamics. The pacemaker 20 can be programmed for mild, moderate, or severe heart disease.

Some embodiments of the device function interactively and in a pulsatile manner. Inflation and deflation of the jacket 12 cam be controlled electronically according to cardiac parameters. In conventional systems, fluid is added or subtracted mechanically by hand. Some embodiments of the invention include a device that is completely implantable, there is no interface with blood components that could cause coagulopathy or related morbidity, the patient can be completely ambulatory and physically active with the device implanted thus contributing to the quality of life, and expensive external monitoring to adjust the compression pressure is not required. Some embodiments of the invention respond to changing hemodynamics, which are constantly monitored. Embodiments of the invention are also cost effective in terms of initial insertions costs, subsequent hospitalizations, and follow-up costs.

Various features and advantages of the invention are set forth in the following claims.

1. A cardiac assist device fully implantable within a patient to assist a heart, the cardiac assist device comprising:
a cardiac jacket that wraps around at least a portion of the heart;
a fluid reservoir coupled to the cardiac jacket via an inflow canal and an outflow canal;
a pump that provides fluid to the cardiac jacket from the fluid reservoir;
a motor coupled to the pump, a speed of the motor controlling a fluid volume in the cardiac jacket; and
a pacemaker coupled to the motor, the pacemaker controlling the speed of the motor based on at least one cardiac parameter.

2. The cardiac assist device of claim 1 wherein the cardiac jacket, the fluid reservoir, the pump, the motor, and the pacemaker are implantable and do not require any external ports.

3. The cardiac assist device of claim 1 wherein none of the cardiac jacket, the fluid reservoir, the pump, the motor, and the pacemaker interface with a patient's blood.

4. The cardiac assist device of claim 1 wherein the cardiac jacket includes a non-distensible layer and a compressible layer.

5. The cardiac assist device of claim 4 wherein the non-distensible layer is an external layer and the compressible layer is an internal layer.

6. The cardiac assist device of claim 5 wherein the internal layer includes shape memory wires positioned at least one of vertically and horizontally.

7. The cardiac assist device of claim 5 wherein the internal layer includes carbon nanotubes.

8. The cardiac assist device of claim 7 wherein the carbon nanotubes transmit at least one cardiac parameter via telemetry.

9. The cardiac assist device of claim 1 wherein the cardiac jacket includes at least two compartments.

10. The cardiac assist device of claim 9 wherein the at least two compartments include at least two of a left ventricular compartment, a right ventricular compartment, an atrial compartment, and a ventricular compartment.

11. The cardiac assist device of claim 10 wherein the left ventricular compartment and the right ventricular compartment are controlled independently with a delay.

12. The cardiac assist device of claim 1 I wherein the delay is about 30 milliseconds to about 34 milliseconds.

13. The cardiac assist device of claim 1 wherein at least one of the inflow canal and the outflow canal includes a check valve.

14. The cardiac assist device of claim 1 wherein a fluid space in the cardiac jacket is primed with fluid at about zero pressure.

15. The cardiac assist device of claim 1 wherein at least one of the cardiac jacket and the fluid reservoir is constructed of at least one layer of material that is leak-proof, impermeable, and self-sealing.

16. The cardiac assist device of claim 1 wherein a prime volume of the fluid reservoir is predetermined based on a size of the patient and a degree of left ventricular dysfunction.

17. The cardiac assist device of claim 16 wherein the fluid reservoir includes an additional fluid volume to adjust hemodynamics, the additional fluid volume being about 10 percent to about 20 percent of the prime volume.

18. The cardiac assist device of claim 16 wherein a compression fluid volume is added to the prime volume, the compression fluid volume depending on a desired systolic pressure.

19. The cardiac assist device of claim 1 wherein the pump includes a length of about three centimeters to about four centimeters and a diameter of about five centimeters.

20. The cardiac assist device of claim 1 wherein the pump is constructed of material including high purity thermoplastic.

21. The cardiac assist device of claim 1 wherein the pump includes at least one impellar and a shaft constructed of a material including ceramic.

22. The cardiac assist device of claim 1 and further comprising a connector to change rotation of the motor to linear movement of the pump, the connector including a wheel, the wheel having a diameter approximately equal to a displacement of a shaft of the pump.

23. The cardiac assist device of claim 1 wherein the motor is a servo brushless direct current motor.

24. The cardiac assist device of claim 1 and further comprising a battery connected to the motor, the battery being externally recharged by radio frequency through a coil external to the patient.

25. The cardiac assist device of claim 1 wherein the motor includes a length of about 17 millimeters and a diameter of about 10 millimeters.

26. The cardiac assist device of claim 1 wherein the motor operates according to at least one of a normal voltage of about three Volts to about six Volts, a power output of about two Watts, an efficiency of about 69 percent, a rotor inertia of about 0.6 grams centimeters squared, and a maximum recommended speed of about 1000 revolutions per minute.

27. The cardiac assist device of claim 1 wherein a pressure in the cardiac jacket is about 100 millimeters of mercury when a compression volume is about 70 cubic centimeters.

28. The cardiac assist device of claim 1 wherein the motor and the pump at least one of deliver and remove 100 cubic centimeters of fluid per second to the cardiac jacket.

29. The cardiac assist device of claim 1 wherein the pacemaker regulates compression of the heart by the cardiac jacket, the pacemaker causing compression during a systolic phase in order to increase systolic blood pressure.

30. The cardiac assist device of claim 29 wherein the pacemaker regulates decompression of the heart by the cardiac jacket, the pacemaker causing decompression during a diastolic phase in order to at least partially assist the heart in the diastolic phase.

31. The cardiac assist device of claim 1 wherein synchronization by the pacemaker is based on at least one of dual-mode, dual-pacing, dual-sensing pacing, biventricular pacing, and three-chamber synchronization pacing.

32. The cardiac assist device of claim 1 wherein the pacemaker regulates a pulsation ratio of one of one to one, one to two, one to three, and one to four.

33. The cardiac assist device of claim 1 wherein a lower pulsation ratio extends use of a rechargeable battery powering the motor to between about two hours and about six hours.

34. The cardiac assist device of claim 1 wherein the pacemaker includes a processor that determines left ventricular cardiac parameters and right ventricular cardiac parameters.

35. The cardiac assist device of claim 34 wherein the pacemaker includes a processor that determines at least one of left ventricular end diastolic pressure, left ventricular end systolic pressure, right ventricular end diastolic pressure, right ventricular end systolic pressure, left ventricular volume, right ventricular volume, cardiac tension, cardiac output, systolic blood pressure, diastolic blood pressure, and heart rate.

36. The cardiac assist device of claim 1 wherein the pacemaker responds to changes in the at least one cardiac parameter by changing at least one of an inflation rate, a deflation rate, and fluid volume.

37. The cardiac assist device of claim 1 wherein the pacemaker continuously monitors and regulates cardiac hemodynamics in real time.

38. The cardiac assist device of claim 1 wherein the cardiac jacket, the fluid reservoir, the pump, the motor, and the pacemaker are fully implantable subcutaneously in at least one of the left chest, the right chest, and the upper abdomen.

39. The cardiac assist device of claim 1 wherein the pacemaker is programmed for one of mild heart disease, moderate heart disease, and severe heart disease.

40. A method of assisting a heart of a patient, the method comprising:
fully implanting a cardiac assist device within the patient, the cardiac assist device including a cardiac jacket, a fluid reservoir, a pump, a motor, and a pacemaker;
wrapping the cardiac jacket around at least a portion of the heart;
pumping fluid to the cardiac jacket from the fluid reservoir; and
controlling a speed of the motor based on at least one cardiac parameter in order to control a fluid volume in the cardiac jacket.

41. The method of claim 40 and further comprising preventing a patient's blood from interfacing with the cardiac jacket, the fluid reservoir, the pump, the motor, and the pacemaker.

42. The method of claim 40 and further comprising compressing an internal layer of the cardiac jacket and preventing compression of an external layer of the cardiac jacket.

43. The method of claim 42 and further comprising returning the internal layer to an original shape after compressing the internal layer.

44. The method of claim 40 and further comprising transmitting at least one cardiac parameter with carbon nanotubes via telemetry.

45. The method of claim 40 and further comprising wrapping a first compartment of the cardiac jacket around a left ventricle and wrapping a second compartment of the cardiac jacket around a right ventricle.

46. The method of claim 40 and further comprising wrapping a first compartment of the cardiac jacket around ventricles and wrapping a second compartment of the cardiac jacket around atrium.

47. The method of claim 45 and further comprising controlling the first compartment and the second compartment independently with a delay.

48. The method of claim 47 and further comprising delaying pulsation between the first compartment and the second compartment by about 30 milliseconds to about 34 milliseconds.

49. The method of claim 40 and further comprising restricting fluid flow between the fluid reservoir and the cardiac jacket to one direction.

50. The method of claim 40 and further comprising priming a fluid space in the cardiac jacket to about zero pressure.

51. The method of claim 40 and further comprising constructing at least one of the cardiac jacket and the fluid reservoir of at least one layer of material that is leak-proof, impermeable, and self-sealing.

52. The method of claim 40 and further comprising determining a prime volume of the fluid reservoir based on a size of the patient and a degree of left ventricular dysfunction.

53. The method of claim 52 and further comprising providing an additional fluid volume to adjust hemodynamics, the additional fluid volume being about 10 percent to about 20 percent of the prime volume.

54. The method of claim 52 and further comprising adding a compression fluid volume to the prime volume, the compression fluid volume depending on a desired systolic pressure.

55. The method of claim 40 and further comprising constructing the pump of material including high purity thermoplastic.

56. The method of claim 40 and further comprising constructing at least one impellar and a shaft of the pump of a material including ceramic.

57. The method of claim 40 and further comprising changing rotation of the motor to linear movement of the pump with a connector, the connector including a wheel, the wheel having a diameter approximately equal to a displacement of a shaft of the pump.

58. The method of claim 40 and further comprising externally recharging a battery connected to the motor by radio frequency through a coil external to the patient.

59. The method of claim 40 and further comprising operating the motor according to at least one of a normal voltage of about three to about six Volts, a power output of about two Watts, an efficiency of about 69 percent, a rotor inertia of about 0.6 grams centimeters squared, and a maximum recommended speed of about 1000 revolutions per minute.

60. The method of claim 40 and further comprising generating a pressure in the cardiac jacket of about 100 millimeters of mercury when a compression volume is about 70 cubic centimeters.

61. The method of claim 40 and further comprising at least one of delivering and removing 100 cubic centimeters of fluid per second to the cardiac jacket.

62. The method of claim 40 and further comprising regulating compression of the heart, and causing compression during a systolic phase in order to increase systolic blood pressure.

63. The method of claim 40 and further comprising regulating decompression of the heart, and causing decompression during a diastolic phase in order to at least partially assist the heart in the diastolic phase.

64. The method of claim 40 and further comprising synchronizing pulsations based on at least one of dual-mode, dual-pacing, dual-sensing pacing, biventricular pacing, and three-chamber synchronization pacing.

64. The method of claim 40 and further comprising regulating a pulsation ratio of one of one to one, one to two, one to three, and one to four.

65. The method of claim 40 and further comprising reducing a pulsation ratio in order to extend use of a rechargeable battery powering the motor to between about two hours and about six hours.

66. The method of claim 40 and further comprising determining left ventricular cardiac parameters and right ventricular cardiac parameters.

67. The method of claim 66 and further comprising determining at least one of left ventricular end diastolic pressure, left ventricular end systolic pressure, right ventricular end diastolic pressure, right ventricular end systolic pressure, left ventricular volume, right ventricular volume, cardiac tension, cardiac output, systolic blood pressure, diastolic blood pressure, and heart rate.

68. The method of claim 40 and further comprising responding to changes in the at least one cardiac parameter by changing at least one of an inflation rate, a deflation rate, and fluid volume.

69. The method of claim 40 and further comprising continuously monitoring and regulating cardiac hemodynamics in real time.

70. The method of claim 40 and further comprising fully implanting the cardiac jacket, the fluid reservoir, the pump, the motor, and the pacemaker subcutaneously in at least one of the left chest, the right chest, and the upper abdomen.

71. The method of claim 40 and further comprising programming the pacemaker for one of mild heart disease, moderate heart disease, and severe heart disease.
